(19) United States
(12) Patent Application Publication
(10) Pub. No.: US 2023/0102689 A1
(43) Pub. Date: Mar. 30, 2023

(54) MOVEMENT CORRECTION SYSTEM AND METHODS

(71) Applicant: DE ORO DEVICES, INC., San Luis Obispo, CA (US)

(72) Inventors: Sidney COLLIN, San Luis Obispo, CA (US); Adam SCHWARTZ, San Luis Obispo, CA (US); William THOMPSON, San Luis Obispo, CA (US)

(21) Appl. No.: 17/911,385

(22) PCT Filed: Mar. 17, 2021
(86) PCT No.: PCT/US2021/022797

§ 371 Date: Sep. 13, 2022

Related U.S. Application Data
(60) Provisional application No. 62/991,310, filed on Mar. 18, 2020.

(51) Int. Cl. G09B 19/00; G16H 20/30; G16H 40/40; G16H 40/63

(52) U.S. Cl. CPC G09B 19/003; G16H 20/30; G16H 40/40; G16H 40/63

(57) ABSTRACT

Provided are systems and methods for improving movement irregularities.

### CROSS-REFERENCE

This application claims the benefit of U.S. Provisional Application No. 62/991,310, filed Mar. 18, 2020, which application is incorporated herein by reference.

### BACKGROUND

Gait irregularities, such as freezing of gait (FOG) or akinesia, can occur in people who suffer from movement disorders and/or neurological diseases such as Parkinson's disease, cerebral palsy, and multiple sclerosis. Particularly for sufferers of Parkinson's disease, gait irregularity typically occurs when walking in familiar surroundings and in tight corners or doorways, and typically occurs as a brief unexpected inability to walk. This not only interrupts daily life, but often causes accidents, such as falls.

### SUMMARY

Provided herein are embodiments of a device for treatment of a movement disorder, the device comprising: a metronome having a rate; and a light source configured to project on an external surface. In some embodiments, the device further comprises an accelerometer. In some embodiments, the accelerometer is configured as a step counter. In some embodiments, a ratio of a number of steps recorded by the step counter over a predetermined interval of time is used to calibrate the rate of the metronome. In some embodiments, a fall event is registered when the accelerometer senses an acceleration which exceeds a predetermined threshold. In some embodiments, an alert is produced subsequent to the fall event being registered. In some embodiments, the device further comprises at least one audio transducer, and the alert comprises an auditory signal emitted from the at least one audio transducer.

In some embodiments, the device further comprises a belt clip. In some embodiments, the belt clip comprises a first portion and a second portion, wherein the second portion is biased towards the first portion to retain the device onto an article worn by a user. In some embodiments, the second portion is biased towards the first portion by a spring. In some embodiments, the belt clip is rotatably relative to a body of the device. In some embodiments, a first mating disposed on the body of the device comprises a plurality of teeth to engages a second mating surface disposed on the clip to provide a rotatable engagement allowing for selective positioning of the body of the device relative to the clip. In some embodiments, the second mating surface comprises a plurality of teeth to engage with the plurality of teeth of the first mating surface. In some embodiments, the plurality of teeth of the first mating surface are provided on a circular protrusion and about an axis of rotation by which the clip rotates relative to the body of the device. In some embodiments, an angle between the belt clip and a body of the device is adjustable. In some embodiments, a means for adjusting the angle between the belt clip and a body of the device comprises an adjustable hinge formed by a partially cylindrical surface having a plurality indentations which are engaged by a locking tab.

In some embodiments, the device further comprises a communication module configured to transmit audio signals from the metronome to an external audio device. In some embodiments, the external audio device comprises wireless hearing aids. In some embodiments, external audio device comprises wireless headphones. In some embodiments, the device further comprises a microphone, wherein the microphone is configured to receive voice commands from a user to changes settings of the device. In some embodiments, the rate of the metronome is determined by an elapsed time between peaks of at least two audio events received by the microphone.

In some embodiments, the external surface is a walking surface. In some embodiments, the light source is a laser. In some embodiments, the laser is provided on a rotatable platform. In some embodiments, the laser is configured to project one or more lines onto the external surface. In some embodiments, the one or more lines are configured to be perpendicular to the line of site of a body of the cueing device. In some embodiments, the external surface is a walking surface.

In some embodiments, the device further comprises a tilt switch configured to deactivate the light source when the device is angled such that a projection of the light source exceeds a predetermined angle relative to a direction of experienced gravitational force. In some embodiments, the tilt switch comprises a gyroscopic sensor. In some embodiments, the predetermined angle is 60 degrees.

In some embodiments, the device further comprises a volume control. In some embodiments, wherein the volume control is a potentiometer. In some embodiments, the volume control comprises two or more buttons. In some embodiments, the device further comprises a metronome rate control. In some embodiments, the metronome rate control is a potentiometer. In some embodiments, the metronome rate control comprises two or more buttons.

Provided herein are embodiments of a method of preventing an irregular movement in a user, the method comprising: setting a rate of a metronome; emitting an audio signal to the user, the audio signal corresponding with the rate of the metronome; and projecting a visual cue, wherein the visual cue is projected onto a walking surface in front of the user.

In some embodiments, the method further comprises a step of counting steps of the user over a predetermined period of time, and wherein the step of setting the rate of the metronome further comprises automatically setting the rate of the metronome based on a number of steps counted over the predetermined period of time. In some embodiments, the method further comprises recording a time elapsed since recordation of a step and recording an irregular movement event if the time lapsed exceeds a predetermined elapsed. In some embodiments, the method further comprises a step of resetting the rate of the metronome subsequent to recording the irregular movement event. In some embodiments, the method further comprises a step of emitting a sequence of audio signals subsequent to recording the irregular movement event.

In some embodiments, wherein the sequence of audio signals comprises one or more voice instructions directed to assisting the user in overcoming the irregular movement event. In some embodiments, the step of emitting an audio signal to the user further comprises wirelessly transmitting the audio signal to an audio device. In some embodiments, the method further comprises receiving one or more voice commands. In some embodiments, the step of setting the rate of the metronome is based upon receiving the one or more voice commands. In some embodiments, the step of setting the rate of the metronome further comprises analyzing a time elapsed between peaks of at least two audio signals received and setting the rate of the metronome to correspond to elapsed time between the at least two audio signals received.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 is a perspective view of an embodiment of a cueing device.

FIG. 2 is a perspective exploded view of cueing device of FIG. 1.

FIG. 3 is a block diagram of a control system of the cueing device of FIG. 1.

FIG. 4 is a perspective view of the cueing device of FIG. 1 mounted to a walker.

FIG. 5 is a perspective view of the cueing device of FIG. 1 mounted to a cane.

FIG. 6A is a perspective view of an embodiment of a cueing device.

FIG. 6B is a perspective view of the cueing device of FIG. 6A mounted to a belt of a user.

FIG. 7A is a perspective view of an embodiment of a cueing device.

FIG. 7B is a perspective view of the curing device of FIG. 7A mounted to a wrist of a user.

FIG. 8A is a perspective view of an embodiment of a cueing device.

FIG. 8B is a perspective view of an embodiment of a cueing device.

### DETAILED DESCRIPTION

Provided herein are systems and apparatuses to mitigate FOG or other walking disorders may include a cueing device comprising an activation module and a cueing module. The activation module may be disposed adjacent a handlebar of an assistive mobility device such as a walker or cane such that the activation module is within easy reach of a user's fingers. The activation module may be configured to activate an audible cue and a visual cue.

The audible cue may be a click or chirp from a metronome and the visual cue may be a line projected onto a walking surface by a laser light source. The cueing module may be adjustably coupled to a lower portion of a forward leg or back leg of the walker or a shaft of the cane. The cueing module may include a laser light source, a metronome, and a speaker. A potentiometer may control a cadence rate of the metronome and another potentiometer may control the volume output of the speaker. The laser light source may be adjustable relative to a longitudinal axis of the cueing module such that the position of the projected line relative to the user may be adjusted. An adjustable length cable may couple the activation module to the cueing module such that the modules are in electronic communication.

In use, the device to mitigate FOG may be coupled to an assistive mobility device with the activation module positioned adjacent the user's fingers and the cueing module above the walking surface. The device may be powered on by activating a power switch on the cueing module. The cadence rate of the metronome and the speaker volume may be adjusted to a suitable rate and volume by rotating a corresponding knob on the cueing module. The user may activate either the metronome or the laser light source or both by depressing a corresponding button on the activation module prior to initiating walking. Alternatively, the user may activate either the metronome or the laser light source or both after encountering a FOG episode.

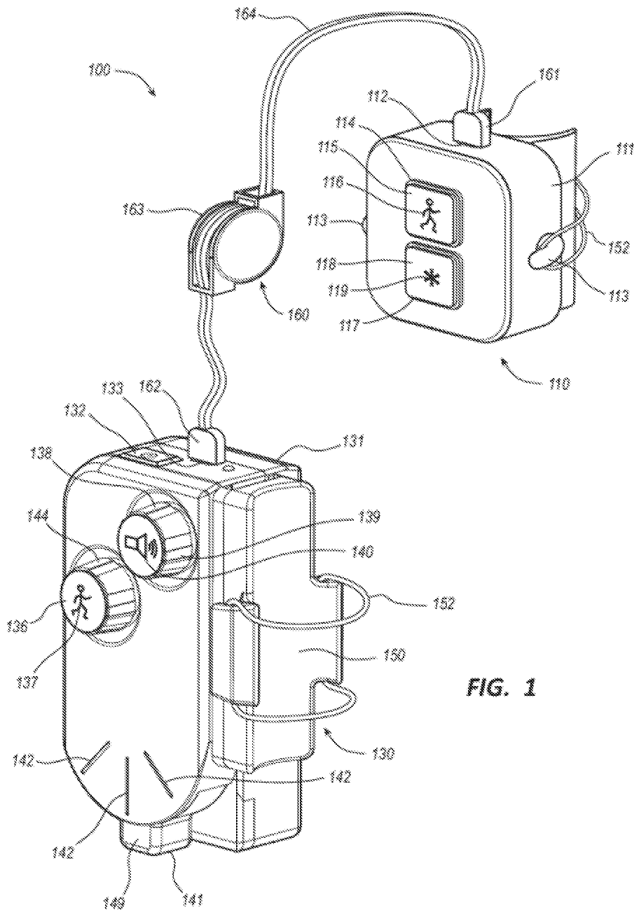

FIGS. 1-8B illustrate different views of several cueing devices and related components. In certain views each device may be coupled to, or shown with, additional components not included in every view. Further, in some views only selected components are illustrated, to provide detail into the relationship of the components. Some components may be shown in multiple views but not discussed in connection with every view. Disclosure provided in connection with any figure is relevant and applicable to disclosure provided in connection with any other figure or embodiment.

FIGS. 1-5 depict some embodiments of a cueing device 100. In the illustrated embodiments, the cueing device 100 includes three broad groups of components; each group may have numerous subcomponents and parts. The three broad component groups are: an activation module 110, a cueing module 130, and a cable 160.

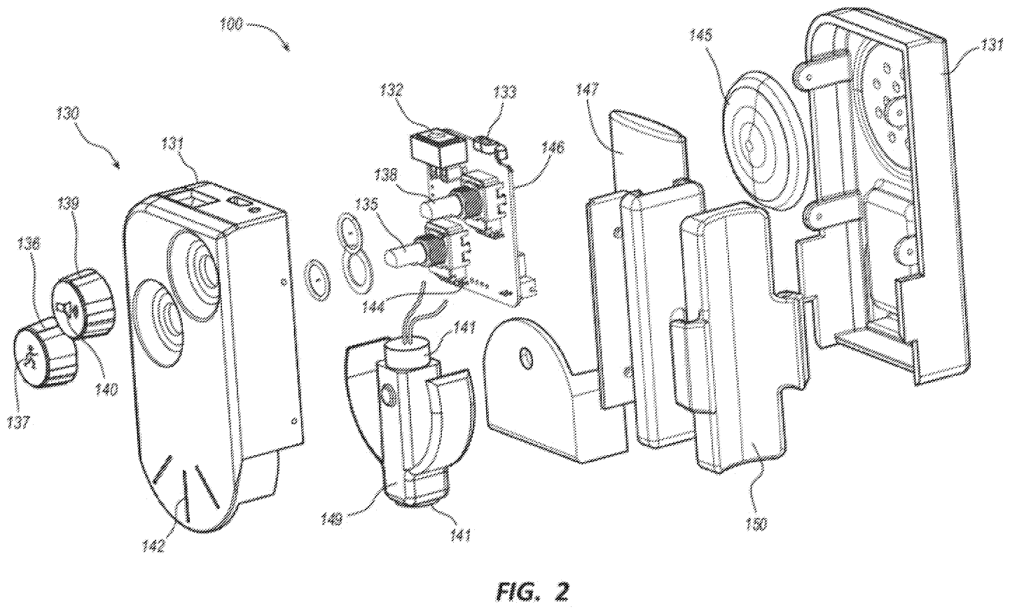

As illustrated in FIGS. 1-2, the activation module 110 includes a housing 111, an audible cueing activation switch or metronome activation switch 114, a visual cueing activation switch or laser light source activation switch 117, and a universal serial bus (USB) port 112. In some embodiments, the activation module 110 may include a music cueing activation switch. The housing 111 may be in a shape of a square box having side dimensions of about 1.25 inches and a depth dimension of about 0.75 inch or suitable dimensions that are within the scope of this disclosure. In some embodiments, the housing 111 may be formed in the shape of a circle, a rectangle, an oval, or other geometries that are likewise within the scope of this disclosure. The housing 111 may include a front portion and a back portion that are coupled using any suitable technique, such as snap fit, friction fit, welding, bonding, etc. The housing 111 can be formed from any suitable plastic material, such as polycarbonate, polypropylene, acrylonitrile butadiene styrene, acrylic, polyurethane, etc., or metal material, such as steel, aluminum, etc. The housing 111 may be formed using any suitable manufacturing method, such as machining, thermal injection molding, reactive injection molding, thermoforming, stamping, casting, 3-D printing, multi-jet fusion, etc.

The audible cueing activation switch 114 and the visual cueing activation switch 117 are disposed within the housing 111. The switches 114, 117 are configured to activate a metronome 144 and a laser light source 141 of the cueing module 130, respectively. The switches 114, 117 may each include a button 115, 118 disposed within the front portion of the housing 111. The buttons 115, 118 may be configured to be depressed by a user to engage the switches 114, 117 when activation of the metronome 144 and/or laser light source 141 is desired. An audible cueing activation indicium 116 may be disposed on the button 115 or adjacent the button 115 on the housing 111. The indicium 116 may represent a walking person or any suitable image. A visual cueing activation indicium 119 may be disposed on the button 118 or adjacent the button 118 on the housing 111. The indicium 119 may represent a universal accepted symbol for a laser (e.g., starburst) or any suitable image. In some embodiments, an indicator light may be disposed adjacent the buttons 115, 118 to provide indication of activation of the audible cueing activation switch 114 and/or the visual cueing activation switch 117. In some embodiments, the activation module 110 may be in electronic communication with an external control device such as a smart phone, tablet, etc. using any suitable wireless local area networking technique where the external control device is used to activate the metronome 144 and the laser light source 141.

The USB port 112 is disposed on a side of the housing 111. The USB port 112 is configured to couple with a USB connector 161 of the cable 160 such that the activation module 110 and the cueing module 130 are in electrical communication. In some embodiments, the activation module 110 and the cueing module 130 may be in wireless communication using any suitable wireless local area networking technique. In certain embodiments, the activation module 110 receives power from the cueing module 130 through the USB port 112. In some embodiments, the activation module 110 may include a power source (e.g., batteries).

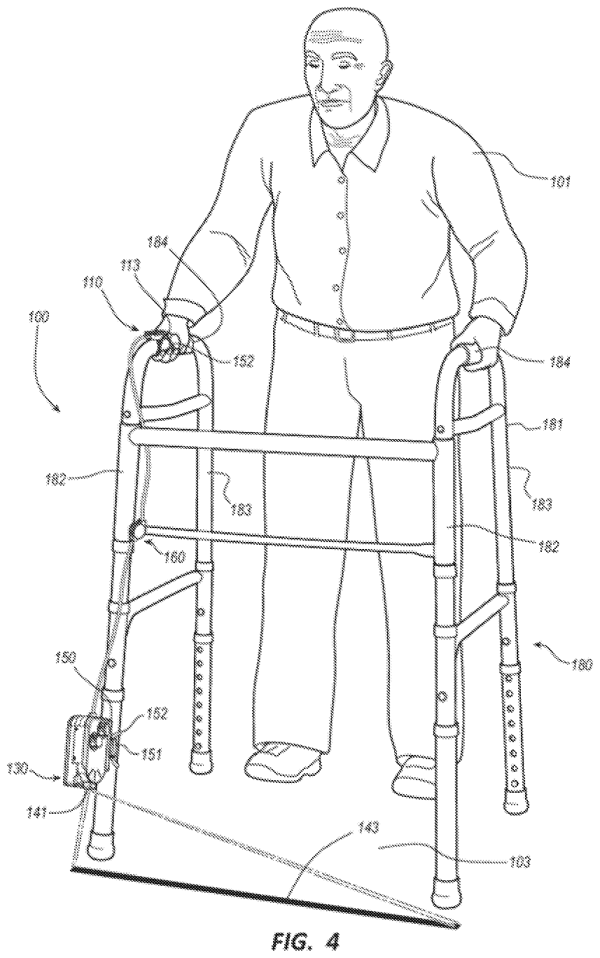

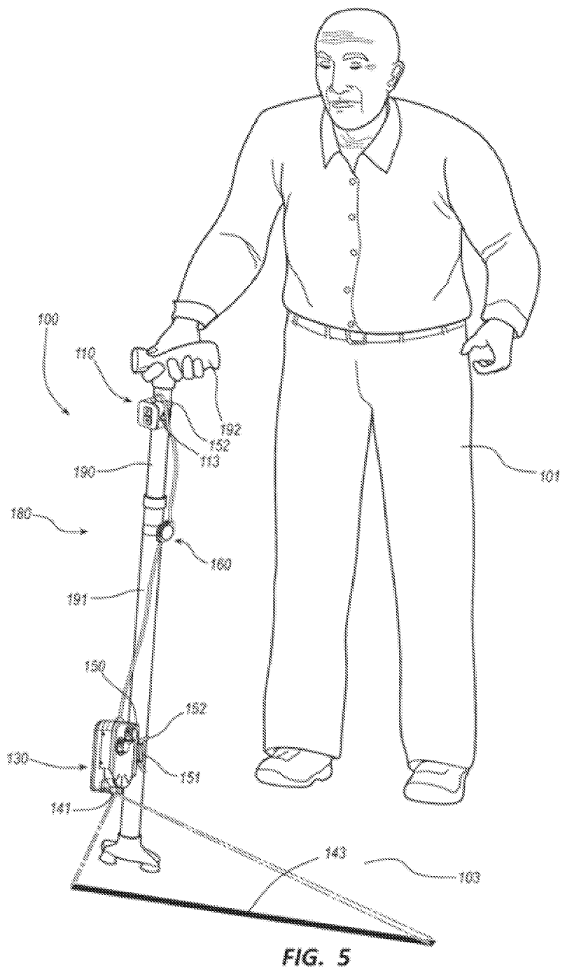

The housing 111 includes mushroom-shaped projections 113 extending from opposite sides of the housing 111. The projections 113 may be configured to facilitate coupling of the activation module 110 to an assistive mobility device 180. (See FIGS. 4 and 5.) An elastomeric member 152 may be coupled to the projections 113 and extend around a portion of the assistive mobility device 180. In some embodiments, the projections 113 may be in the form of hooks, clips, elbows, etc. In some embodiments, the projections 113 may comprise a hook-and-loop material configured to couple with a band of hook-and-loop material.

As depicted in the illustrated embodiments of FIGS. 1-2, the cueing module 130 includes a housing 131, the metronome 144, the laser light source 141, a power switch 132, a power source 147, a processor 146, a metronome potentiometer 135, a speaker 145, a speaker volume potentiometer 138, and a micro USB port 133. The housing 131 may be in a shape of a rectangular box having a length dimension of about 4.00 inches, a width dimension of about 2.00 inches, and a depth dimension of about 1.75 inches or other suitable dimensions that are within the scope of this disclosure. In some embodiments, the housing 131 may be formed in the shape of a circle, a rectangle, an oval, or other geometries that are likewise within the scope of this disclosure. The housing 131 may include a front portion and a back portion that are coupled using any suitable technique, such as snap fit, friction fit, welding, bonding, etc. The housing 131 can be formed from any suitable plastic material, such as polycarbonate, polypropylene, acrylonitrile butadiene styrene, acrylic, polyurethane, etc., or metal material, such as steel, aluminum, etc. The housing 131 may be formed using any suitable manufacturing method, such as machining, thermal injection molding, reactive injection molding, thermoforming, stamping, casting, 3-D printing, multi jet fusion, etc. The housing 131 may include a mounting bracket 150. In some embodiments, the housing 131 may include a slide disposed on a side of the housing 131. The slide may be configured to slidingly engage with the mounting bracket 150.

The metronome 144 is disposed within the housing 131 and electronically coupled to the audible cueing activation switch 114 of the activation module 110 via the cable 160. The metronome potentiometer 135 is coupled to the metronome 144 and configured to adjust a cadence rate output of the metronome 144. The metronome potentiometer 135 may be configured to adjust the cadence rate of the metronome 144 between about zero and about 250 beats per minute, between about 10 and about 100 beats per minute, and about 20 and about 100 beats per minute. A metronome potentiometer knob 136 is coupled to the metronome potentiometer 135 and is configured to be engaged by a user to adjust the cadence rate. An indicium 137 may be disposed on the knob 136 or adjacent the knob 136 on the housing 131. The indicium 137 may be an image of a walking person or other suitable images that are within the scope of this disclosure.

The speaker 145 may be disposed within the housing 131. The speaker 145 may be in electronic communication with the metronome 144 and the speaker volume potentiometer 138. The speaker volume potentiometer 138 can be configured to adjust an output volume of the speaker 145 from about 50 decibels to about 65 decibels. The output sounds from the metronome 144 through the speaker 145 may be pings, blips, chimes, dings, clicks, chirps, claves, agogos, etc. A speaker volume potentiometer knob 139 is coupled to the metronome potentiometer 138 and is configured to be engaged by a user to adjust the output volume of the speaker 145. An indicium 140 may be disposed on the knob 139 or adjacent the knob 139 on the housing 131. The indicium 140 may be an image of a standard speaker symbol or other suitable images that are within the scope of this disclosure.

In some embodiments, the metronome 144 may be in communication with an external device such as headphones, ear buds, smart phone, tablet, hearing aids, etc. using any suitable wireless local area networking technique. In some embodiments, headphones, earbuds, or hearing aids may be connected to the system via a Bluetooth connection. Wireless connection allows a user or patient to utilize auditory cues without drawing attention to their use of the system.

In some embodiments, the system may utilize a panning or spatial auditory cues. The panning of the auditory cues may coordinate with a particular foot which the patient or user is being instructed to step with. For example, auditory cues may be panned left to signal a step with the left foot and panned right to signal a step with the right foot. Audio may be fully panned to one side or headphone or partial panned to one side or headphone. In some embodiments, the audio mix will provide an audio cue to with an effect of spatial positioning in front of user, such that the user feels as if they are stepping toward the audio cue. Spatial audio mixing may include the use of delays, panning, and reverb effects to position the audio cues in a simulated 3-dimensional environment.

In some embodiments, the speaker 145 may be configured for output of music or recorded sounds configured to provide a rhythm or beat. The music or recorded sounds may be stored in memory of the processor 146; wirelessly communicated to the processor 146 from an external device such as a smartphone, tablet, etc. using any suitable local area networking technique; or stored in memory of an external memory storage device such as a USB memory stick or flash drive.

In the illustrated embodiment, the laser light source 141 is disposed at a distal portion of the cueing module 130. The laser light source 141 may include a laser diode configured to emit a red, orange, red-orange, blue, green, yellow, violet, etc. laser beam. As depicted in FIGS. 4 and 5, the laser beam may be projected onto a walking surface 103 as a projected line 143. In certain embodiments, the laser light source 141 may project two, three, four, or more projected lines 143. Multiple projected lines may be projected parallel to one another. The projected line 143 may be in the form of a solid line, a segmented or dashed line, a dotted line, or any suitable form that provides cueing for a user. In some embodiments, the cueing module 130 may include a laser light source potentiometer configured to adjust an intensity of the laser light source 141 such that the projected line 143 is visible by the user in darkened as well as lighted surroundings. In some embodiments, the cueing module 130 may include a light sensor that is configured to automatically adjust the intensity of the laser light source 141 dependent upon the lighting of the surroundings.

In some embodiments, the projected line 143 may be in the form of a foot outline or a shoe outline. In some embodiments, the projection may correspond to a left side or right side projection. For example, the projection may appear as a left shoe outline or a right shoe outline. In some embodiments, the oriented projection such as a left side or right side projection may correlate with an instruction for the user to step with the corresponding foot.

As depicted in of FIGS. 1-2, the laser light source 141 may be disposed within a holder 149 and configured to be rotatably adjusted relative to a central longitudinal axis of the cueing module 130. The laser light source 141 may be in a neutral position when the laser light source 141 is aligned with the central longitudinal axis of the cueing module 130. The laser light source 141 may be in a forward position when it is rotationally adjusted forwardly up to about 80 degrees such that the laser light source 141 may project forward of the central longitudinal axis. The laser light source 141 may be in a rearward position when it is rotationally adjusted rearwardly up to about 80 degrees such that the laser light source 141 may project rearward of the central longitudinal axis. The position of the laser light source 141 may be indicated by a position indicium 142. The position indicium 142 may be of any suitable form, such as a protruding line, a groove, a printed line, etc.

The power switch 132 may be disposed at a proximal end of the cueing module 130 or at any suitable surface. The power switch 132 may be in the form of a rocker switch where the cueing module 130 and the activation module 110 are powered on when the power switch 132 is depressed toward one side and powered off when the power switch 132 is depressed toward an opposite side. In some embodiments, the power switch 132 may be in the form of any suitable on/off type of switch, such as a pushbutton, toggle, motion sensor, etc. The micro USB port 133 may be disposed at a proximal end of the cueing module 130 or at any suitable location. The micro USB port 133 may be configured to receive a micro USB connector 162 of the cable 160. The power source 147 may be disposed within the housing 131. The power source 147 may be a battery pack of one, two, three, or more rechargeable batteries. The power source 147 may be recharged through the micro USB port 133 and the cable 160 when the cable 160 is coupled to the cueing module 130 at one end and a power supply at the opposite end. Certain components of the cueing module 130 may be included in the activation module 110. For example, the power switch 132, the metronome potentiometer 138, the speaker volume potentiometer 138, etc. may be included in the activation module 110. In some embodiments, the potentiometers to set the metronome rate and speaker volume are slider potentiometers. In some embodiments, the metronome rate and speaker volume are instead set by two or more buttons.

FIG. 1 depicts the cable 160 of the illustrated embodiment of the cueing device 100. The cable 160 is shown to include the USB connector 161, the micro USB connector 162, an adjustable member 163, and an elongate insulated wire 164. The cable 160 is configured to provide electrical communication between the activation module 110 and the cueing module 130. The USB connector 161 is disposed at one end of the insulated wire 164 and configured to couple with the USB port 112 of the activation module 110. The USB connector 161 is also configured to couple with an external device such as a battery charging source, an external control device (e.g., smart phone), etc. The micro USB connector 162 is disposed at an opposite end of the insulated wire 164 and configured to couple with the micro USB port 133 of the cueing module 130. The adjustable member 163 is coupled to the insulated wire 164 and configured to automatically adjust a length (longer or shorter) of the cable 160 as the distance between the activation module 110 and the cueing module 130 changes. The adjustable member 163 may be configured to remove slack of the cable 160 to prevent the cable 160 from being inadvertently snagged when the cueing device 100 is in use. In the depicted embodiment, the adjustable member 163 is in the form of a retractable reel. In other embodiments, the cable 160 may include any suitable type of adjustable member 163, automatic or manual, such as a ratchet mechanism, a buckle, etc.

In some embodiments, the cueing device 100 may include sensors configured to monitor a user's walking speed, number of steps taken, number of FOG events, time utilization of the cueing device 100, etc. The sensors may be disposed in either the activation module 110 and/or the cueing module 130. Alternatively, the sensors may be couple to the user and electronically coupled to the cueing device 100 using hardwire or wireless communication techniques. Data collected by the sensors may be stored in a memory of the cueing device 100 or a removable memory member and subsequently uploaded to a computer, smart phone, etc. for analysis. Alternatively, the data may be uploaded to a computer, smart phone, etc. from the sensors on a real time basis using hardwire or wireless communication techniques.

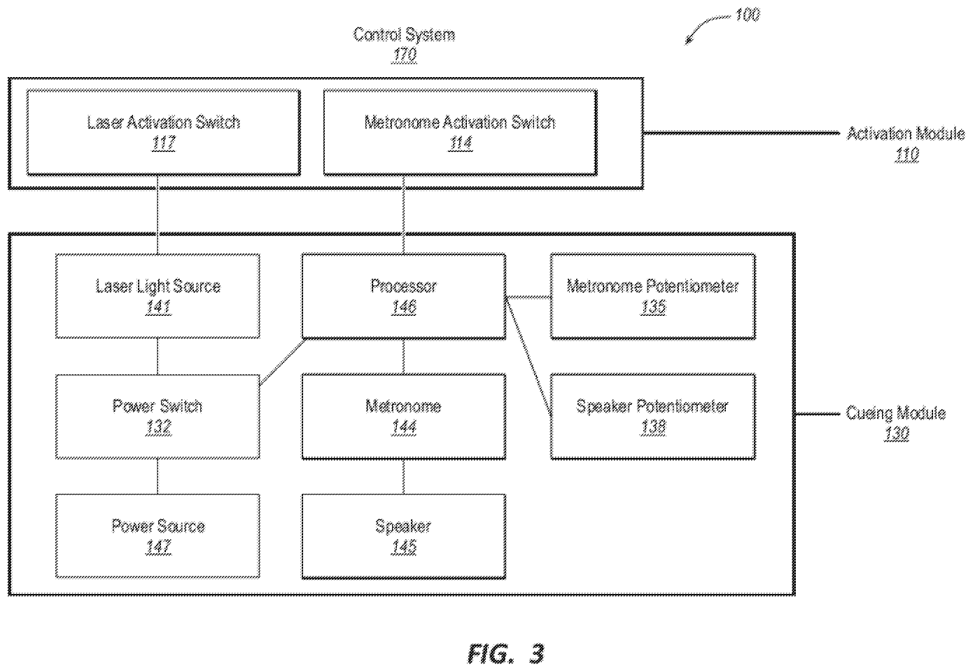

FIG. 3 is a block diagram of a control system 170 of the cueing device 100. The control system 170 includes various components of the activation module 110 and the cueing module 130 as previously described. FIG. 3 illustrates the activation module 110 including the metronome activation switch 114 and the laser activation switch 117. The laser activation switch 117 is in electrical communication with the laser light source 141 of the cueing module 130. When the laser activation switch 117 is actuated by the user, the laser light source 141 is activated. The metronome activation switch 114 is in electrical communication with the processor 146 of the cueing module 130. The processor 146 may be in electronic communication with the metronome 144. In some embodiments, the processor 146 and the metronome 144 may be an integrated unit. The metronome potentiometer 135 is in electronic communication with the processor 146 and the metronome 144. The speaker volume potentiometer 138 is in electronic communication with the processor 146 and the speaker 145. The power source 147 is in electrical communication with the power switch 132 such that when the power switch 132 is actuated, power can be supplied to the laser light source 141, the processor 146, the metronome 144, and the speaker 145.

FIG. 4 illustrates the cueing device 100 coupled to an assistive mobility device 180. In the illustrated embodiment of FIG. 4, the assistive mobility device 180 is in the form of a walker 181. The cueing device 100 is shown coupled to the walker 181 with the cable 160 extending between the modules 110, 130. The walker 181 includes a pair of forward legs 182, a pair of rearward legs 183, and a pair of handle bars 184 configured to be gripped by a user 101. As illustrated, the activation module 110 is adjustably coupled to an upper portion of the walker 181 adjacent the left or right handlebar 184 such that the activation module 110 is within easy reach of a finger of the user 101. The activation module 110 may be coupled to the walker 181 using the mushroom-shaped projections 113 and the elastomeric member 152. Other techniques of coupling, such as hook-and-loop bands, adhesive, buckled straps, etc., are within the scope of this disclosure.

The cueing module 130 can be adjustably coupled to a lower portion of the left or right forward leg 182 of the walker 181. In some embodiments, the cueing module 130 may be adjustably coupled to a lower portion of a left or right back leg of the walker 181. The cueing module 130 may be coupled to the walker 181 using the mounting bracket 150. The mounting bracket 150 may be coupled to the walker 181 through use of the elastomeric member 152 coupled to hooks 151 disposed on sides of the mounting bracket 150. Other techniques of coupling, such as hook-and-loop bands, adhesive, buckled straps, etc., are with the scope of this disclosure.

In some embodiments, the body of the housing comprises a surface having a radial curvature adapted to engage with a walker, cane, or other cylindrical member of an assistive mobility device. In some embodiments, the body of the housing comprises a surface having a curvature or outline adapted to with a non-cylindrical member of an assistive mobility device. In some embodiments, said surface comprises a concave curvature. In some embodiments, the surface adapted to engage with an appendage or body surface of a user. In some embodiments, said surface comprises a concave curvature. In some embodiments, the surface adapted to engage with an assistive mobility device comprises a material configured to increase friction against the surface of the mobility device and retain the cueing module 130 in place. Example materials may comprise rubber, polymers, foam, or other suitable materials.

The height of the cueing module 130 above the walking surface 103 can be adjusted such that intensity and the length of the projected line 143 can be set to be easily visible by the user 101. The position of the projected line 143 relative to the walker 181 can be adjusted by rotating the laser light source 141 forward to position the projected line 143 forward of the walker 181 such that the user 101 may be prompted to take strides longer than when the laser light source 141 is positioned in the neutral position. The laser light source 141 can be rotated rearwardly to position the projected line 143 rearward of the forward legs 182 such that the user 101 may be prompted to take strides shorter than when the laser light source 141 is positioned in the neutral position. The cueing module 130 can be oriented such that the laser light source 141 projects either to the left or to the right.

FIG. 5 illustrates the cueing device 100 coupled to an assistive mobility device 180. In the illustrated embodiment of FIG. 5, the assistive mobility device 180 is in the form of a cane 190. The cueing device 100 is shown coupled to the cane 190 with the cable 160 extending between the modules 110, 130. The cane 190 includes a shaft 191 and a handle 192 disposed at a proximal end of the shaft 191 and configured to be gripped by the user 101. As illustrated, the activation module 110 is adjustably coupled to an upper portion of the shaft 191 adjacent the handle 192 such that the activation module 110 is within easy reach of a finger of the user 101. The activation module 110 may be coupled to the cane 190 using the mushroom-shaped projections 113 and the elastomeric member 152. Other techniques of coupling, such as hook-and-loop bands, adhesive, buckled straps, etc., are with the scope of this disclosure.

The cueing module 130 can be adjustably coupled to a lower portion of the shaft 191 of the cane 190. The cueing module 130 may be coupled to the cane 190 using the mounting bracket 150. The mounting bracket 150 can include a slide receiver (not shown) configured to slidingly receive a slide 148 of the cueing module 130. The mounting bracket 150 may be coupled to the cane 190 through use of the elastomeric member 152 coupled to hooks 151 disposed on sides of the mounting bracket 150. Other techniques of coupling, such as hook-and-loop bands, adhesive, buckled straps, etc., are with the scope of this disclosure.

The height of the cueing module 130 above the walking surface 103 can be adjusted such that the intensity and the length of the projected line 143 can be set to be easily visible by the user 101. The position of the projected line 143 relative to the cane 190 can be adjusted by rotating the laser light source 141 forward to position the projected line 143 forward of the cane 190 such that the user 101 may be prompted to take strides longer than when the laser light source 141 is positioned in the neutral position and the projected line 143 extends outwardly from the shaft 191. The laser light source 141 can be rotated rearwardly to position the projected line 143 rearward of the shaft 191 such that the user 101 may be prompted to take strides shorter than when the laser light source 141 is position in the neutral position. The cueing module 130 can be oriented such that the laser light source 141 projects either to the left or to the right to facilitate right-handed and left-handed use of the cane 190 by the user 101.

In use, a cueing device can be coupled to the assistive mobility device. An activation module can be coupled adjacent the finger of a user to an upper portion of the assistive mobility device. A cueing module may be coupled to a lower portion of the assistive mobility device. The height of the cueing module relative to a walking surface can be adjusted to allow for easy visualization of a projected line on the walking surface by the user. A cable may be coupled to the activation module at one end and the cueing module at the opposite end such that the modules are in electrical communication. A laser light source may be oriented in the neutral position where the projected line is projected in a plane of the longitudinal axis of the cueing module. Alternatively, the laser light source may be oriented forward or rearward of the longitudinal axis such that the projected line is projected either forward or rearward of the plane of the longitudinal axis.

The user may activate the laser light source by depressing a laser activation button with a finger to project the projected line onto the walking surface. Alternatively, or in parallel with activation of the laser light source, the user may depress the metronome activation button with a finger to activate the metronome. The cadence rate of the metronome can be increased or decreased by rotation of a knob by the user. The output volume of the speaker may be increased or decreased by rotation of a second knob by the user. Following activation of the laser light source and/or the metronome, the user may initiate walking with the aid of the assistive mobility device. Upon experiencing an episode of FOG, the user can be prompted or cued to take a step by the projected image and/or the output of the metronome from a speaker. In some embodiments, the laser light source and/or the metronome may be activated by the user following experiencing a FOG episode such that the user is prompted or cued to take a step.

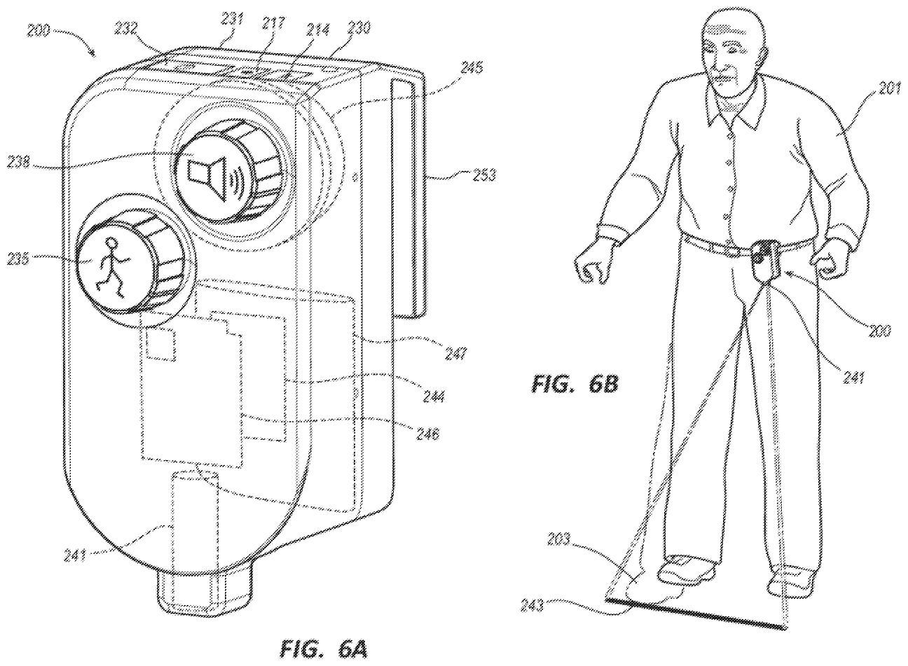

FIGS. 6A-6B depict an embodiment of a cueing device 200 that resembles the cueing device 100 described above in certain respects. Accordingly, like features are designated with like reference numerals, with the leading digit incremented to “2.” For example, the embodiment depicted in FIGS. 6A-6B includes a cueing module 230 that may, in some respects, resemble the cueing module 130 of FIG. 1. Relevant disclosure set forth above regarding similarly identified features thus may not be repeated hereafter. Moreover, specific features of the cueing device 100 and related components shown in FIGS. 1-5 may not be shown or identified by a reference numeral in the drawings or specifically discussed in the written description that follows. However, such features may clearly be the same, or substantially the same, as features depicted in other embodiments and/or described with respect to such embodiments. Accordingly, the relevant descriptions of such features apply equally to the features of the cueing device 200 and related components depicted in FIGS. 6A-6B. Any suitable combination of the features, and variations of the same, described with respect to the cueing device 100 and related components illustrated in FIGS. 1-5 can be employed with the cueing device 200 and related components of FIGS. 6A-6B, and vice versa. This pattern of disclosure applies equally to further embodiments depicted in subsequent figures and described hereafter, wherein the leading digits may be further incremented.

FIGS. 6A-6B depict an embodiment of a cueing device 200. The cueing device 200 may be substantially an integration of the activation module 110 and the cueing module 130 of the cueing device 100. In the depicted embodiment of FIGS. 6A— 6B, the cueing device 200 includes a cueing module 230. The cueing module 230 includes a housing 231, a power switch 232, a laser activation switch 217, a metronome activation switch 214, a metronome potentiometer 235, a speaker volume potentiometer 238, a metronome 244, a processor 246, a speaker 245, a laser light source 241, and a power source 247. The function of each of the listed features of the cueing module 230 is similar to the function of the like feature of cueing module 130 as previously described.

The housing 231 includes a belt hook 253 disposed on a back side of the housing 231. The belt hook 253 is configured to facilitate coupling of the cueing device 200 to a user 201 as depicted in FIG. 6B. The belt hook 253 may extend over a belt, a waistband, a pocket, etc. As illustrated in FIG. 6B, the cueing device 200 may be coupled to a mid-portion of the user 201 such that the cueing device 200 faces forward of the user 201. In an embodiment, the cueing device 200 may be coupled to a user's leg, chest, foot, etc. The laser light source 241 may be configured to project a projected line 243 onto a walking surface 203. The projected line 243 may extend forward of the feet of a user 201 and perpendicular to a direction of travel. A distance from the feet of the user 201 to the projected line 243 may be adjusted by adjusting a projection angle of the laser light source 241 relative to a transverse longitudinal axis of the cueing device 200.

In use, the user 201 may couple the cueing device 200 to a belt, waistband, or pocket using the belt hook 253. The power switch 232 may be activated. The laser activation switch 217 and/or the metronome activation switch 214 may be activated. The metronome potentiometer 235 may be adjusted to set a desired cadence. The speaker volume potentiometer 238 may be adjusted to set a desired output volume from the speaker 245. The angle of projection of the laser light source 241 may be adjusted to set a desired distance of the projected line 243 from the feet of the user 201. In an embodiment, the light source is adjustable about a longitudinal axis of the cueing device 200. The cueing device 200 may cue or prompt the user 201 to take a step when the user 201 encounters a FOG episode.

In some embodiments, the belt hook 253 rotates about the device to accommodate attachment of the device to various areas of the user's clothing. For example, the belt hook 253 could be attached to a pocket of a user's pants and then rotated such that the device is correctly positioned such that the projected line 243 is properly positioned on the walking surface 203. In some embodiments, rotation of the belt hook may be enabled by two sets of teeth provided on the mating surfaces of two opposing protrusions, such that the cueing device 200 can be rotated to a set position and will remain at the appropriate angle while attached to the user 201. In some embodiments, the teeth may be provided about the axis of rotation. In some embodiments, a spring biases the two sets of teeth toward one another to provide consistent engagement. The teeth may have angled sides to form a saw tooth shape. In some embodiments, the engagement of the teeth provides audible and/or tactile feedback to the user. In some embodiments, one side of each tooth may be perpendicular to the plane formed by the radial dial, such that the engaged teeth create a one-way ratchet mechanism. The one-way ratchet mechanism may be configured such that the cueing device rotates counter-clockwise or clockwise relative to the belt hook.

In some embodiments, the belt hook 253 may be a clip. The clip may utilize a means of rotation about the cueing device as described above. In some embodiments, the belt hook may comprise a spring loaded clip. The clip or belt clip may be biased toward the device to clamp onto an article of clothing or accessory, such as a belt. In such embodiments, the user may depress a surface of the clip to bias the clip away from the device, allowing the clip to be attached to an article of clothing. Upon releasing the clip, the spring bias will cause the clip to clamp onto the article of clothing to retain the device in place.

In some embodiments, the clip comprises a first portion and a second portion, wherein the second portion is biased towards the first portion to retain an article of clothing or accessory (such as a belt) between the first portion and the second portion of the clip.

In some embodiments, a belt hook or clip may be detachable. In some embodiments, belt hook or clip may attach and release from a protrusion extending from a surface of the cueing device 200. In some embodiments, the protrusion may be configured to receive a plurality of attachment mechanisms. The attachment mechanisms may be configured to attach to assistive mobility devices, articles of clothing, planar surfaces, curved surfaces, or other means to which the user may desire to attach the cueing device to. The ability of attaching the cueing device to receive multiple attachment mechanisms would allow a user to swap attachment mechanisms as necessary. This may be useful if mobility of the user increases and desires to move the cueing device from an assistive mobility device to an article of clothing.

In some embodiments, the body of the cueing module is adjustable relative to the belt clip. In some embodiments, the portion of the belt clip which attaches to the body of the cueing module may be an adjustable hinge formed by a partially cylindrical surface with multiple indentations which are engaged by a locking tab. In some embodiments, the locking tab is provided on the belt clip. In some embodiments the locking tab is provided on the body of the cueing device. The adjustable angle may allow a user to adjust the distance of the projected line 243 from their body.

In some embodiments, adjustment of the cueing module relative to the belt clip, or vice versa, will produce an audible click and/or tactile engagement. In some embodiments, wherein an adjustable hinge formed by a partially cylindrical surface with multiple indentations which are engaged by a locking tab is provided, the audible click and tactile engagement may be produced by the locking tab riding over the indentations and then engaging into the indentations due to the elasticity of the locking tab biasing the tab into the indentations. In some embodiments, wherein an engagement of two sets of teeth about a rotation of the belt hook may be enabled by two sets of teeth provided about the circumference of a radial surface

In some embodiments, the adjustable angle between the belt hook and body of the cueing device is about 40 degrees to about 90 degrees. In some embodiments, the adjustable angle between the belt hook and body of the cueing device is about 40 degrees to about 50 degrees, about 40 degrees to about 60 degrees, about 40 degrees to about 70 degrees, about 40 degrees to about 80 degrees, about 40 degrees to about 90 degrees, about 50 degrees to about 60 degrees, about 50 degrees to about 70 degrees, about 50 degrees to about 80 degrees, about 50 degrees to about 90 degrees, about 60 degrees to about 70 degrees, about 60 degrees to about 80 degrees, about 60 degrees to about 90 degrees, about 70 degrees to about 80 degrees, about 70 degrees to about 90 degrees, or about 80 degrees to about 90 degrees. In some embodiments, the adjustable angle between the belt hook and body of the cueing device is about 40 degrees, about 50 degrees, about 60 degrees, about 70 degrees, about 80 degrees, or about 90 degrees. In some embodiments, the adjustable angle between the belt hook and body of the cueing device is at least about 40 degrees, about 50 degrees, about 60 degrees, about 70 degrees, or about 80 degrees. In some embodiments, the adjustable angle between the belt hook and body of the cueing device is at most about 50 degrees, about 60 degrees, about 70 degrees, about 80 degrees, or about 90 degrees.

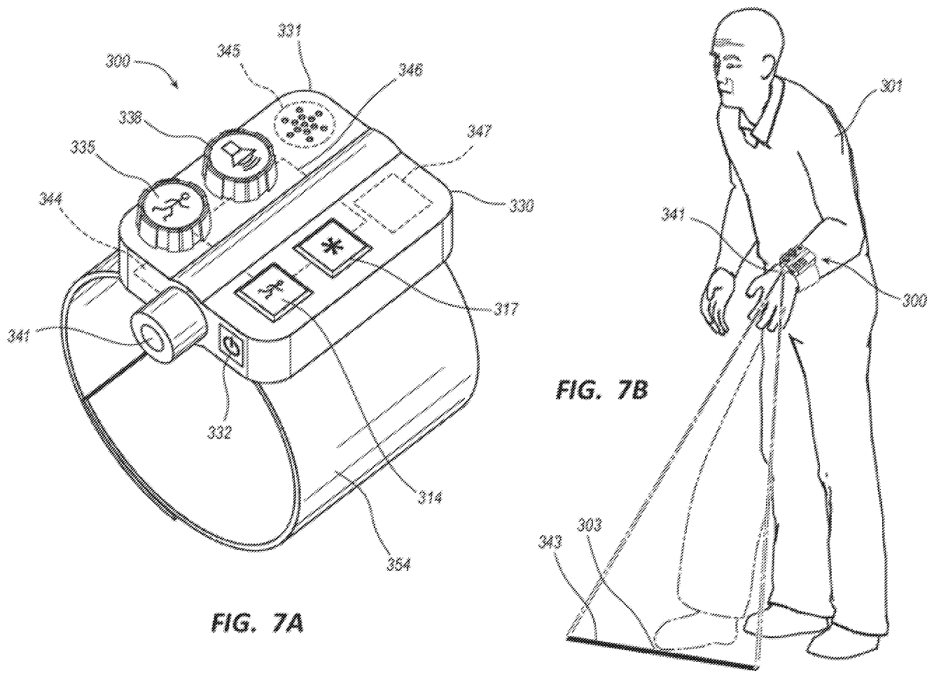

FIGS. 7A-7B depict an embodiment of a cueing device 300. The cueing device 300 may be substantially an integration of the activation module 110 and the cueing module 130 of the cueing device 100. In the depicted embodiment of FIGS. 7A— 7B, the cueing device 300 includes a cueing module 330. The cueing module 330 includes a housing 331, a power switch 332, a laser activation switch 317, a metronome activation switch 314, a metronome potentiometer 335, a speaker volume potentiometer 338, a metronome 344, a processor 346, a speaker 345, a laser light source 341, and a power source 347. The function of each of the listed features of the cueing module 330 is similar to the function of the like feature of cueing module 130 as previously described.

The housing 331 includes a wrist securement band 354 coupled to the housing 331. The securement band 354 is configured to facilitate coupling of the cueing device 300 to a user 301 as depicted in FIG. 7B. The securement band 354 may be any suitable band configured to be releasably secured to the user 101. For example, the securement band 354 may include hook-and loop material, a buckle, a resilient material, etc. The cueing device 300 may be coupled to either the left or right wrist of the user 301 such that the laser light source 341 faces forward of the user 301. The laser light source 341 may be configured to project a projected line 343 onto a walking surface 303. The projected line 343 may extend forward of the feet of the user 301 and perpendicular to a direction of travel. A distance from the feet of the user 301 to the projected line 343 may be adjusted by adjusting an angle of the lower arm relative to the body of the user 301.

In use, the user 301 may couple the cueing device 300 to the wrist using the securement band 354. The power switch 332 may be activated. The laser activation switch 317 and/or the metronome activation switch 314 may be activated. The metronome potentiometer 335 may be adjusted to set a desired cadence. The speaker volume potentiometer 338 may be adjusted to set a desired output volume from the speaker 345. The angle of projection of the laser light source 341 may be adjusted to set a desired distance of the projected line 343 from the feet of the user 301 by adjusting the angle of the lower arm relative to the body of the user 301. The cueing device 300 may cue or prompt the user 301 to take a step when the user 301 encounters a FOG episode.

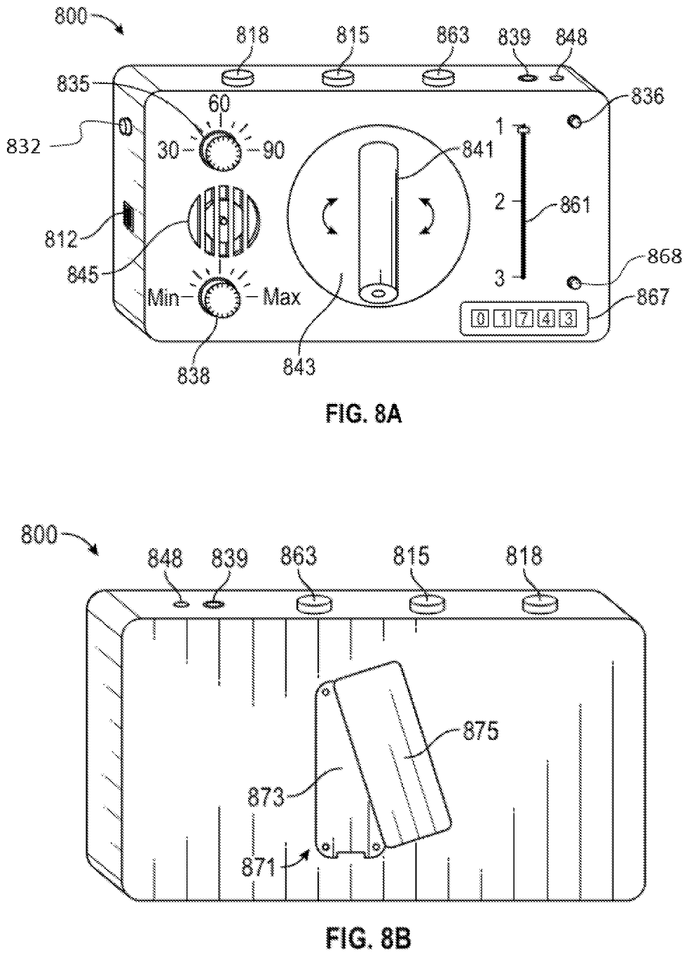

With reference to FIGS. 8A-8B, an embodiment of the movement correction system is depicted. The embodiment shown may comprise a cueing device 800. In some embodiments the cueing device comprises a laser activation button 818 for activating and deactivating laser 841. In some embodiments, laser 841 comprises the visual cueing of the cueing device. Laser 841 may be provided on a rotatable platform 843 to allow a user to adjust the position of the laser relative to the body of the cueing device. In some embodiments, the angle of the laser 841 is adjustable relative to the body of the device 800. This may allow for the projection emitted by the laser to be positioned closer to or further from the user. In some embodiments, adjustment of the laser is possible about two axes, wherein the rotatable platform allows side-to-side adjustment of the laser about a first axis and laser 841 is also adjustable about a second axis to allow front-to-back adjustment toward or away from the device 800 and user.

In some embodiments, the cueing device 800 further comprises a metronome button 815 for activating and deactivating the metronome of the device. In some embodiments, the metronome button may be a metronome rate button, configured to set the metronome rate. The metronome rate button may be depressed and the metronome rate will be set according to an elapsed time interval between depressions. In some embodiments, the metronome rate button must be pressed at least twice to set the metronome rate. In some embodiments the metronome button 815 may be dually function as both a metronome activation button and a metronome rate button. In some embodiments, the metronome is automatically activated when the device is powered on or when the light source is activated.

The device may further comprise a metronome rate control knob 835. In some embodiments, the metronome rate control knob sets the rate of the metronome. The metronome rate may also be set by other means described herein. In some embodiments, audio cues produced by the metronome are emitted via speaker 845. In some embodiments, the cueing device 800 is provided with a headphone output 839 to input a pair of headphones. Insertion of a corresponding input or jack into the headphone output may deactivate speaker 845 or other audio output means disclosed herein. The headphone output may be configured to receive a three pole or four pole input. In some embodiments, the headphone out may receive audio from a microphone when a four pole input when plugged into the headphone output 839. Audio received from a user may comprise voice commands to change settings of the cueing device.

In some embodiments, the cueing device 800 is a wireless compatible device and may wirelessly transmit audio signals to wirelessly coupled audio device. The wireless connection may be a Bluetooth connection. In some embodiments, the cueing device is provided with a wireless connection button 836 to activate a wireless connection. In some embodiments, connection with a wireless device may deactivate transmission of audio to the speaker 845 and/or the headphone output 839. In some embodiments, the wireless connection button incorporates a light source to indicate that wireless connectivity is activated or a wireless device is connected. In some embodiments, a separate light source indicates that wireless connectivity is activated or a wireless device is connected.

In some embodiments, the cueing device 800 comprises a volume control knob 838. The volume control knob may increase or decrease the audio signal sent to the audio output which is active (i.e. the speaker 845, headphone output 839, or Bluetooth output). In some embodiments, turning the volume control knob to a minimum or zero position may deactivate the metronome.

In some embodiments, a step counter display 867 is provided. The step counter may be coupled to an internal step counter or pedometer and display the steps taken. The count of steps taken may represent steps taken over a predetermined time period. For example, steps may be counted from when the device was activated or steps may be counted from when the device was last reset. In some embodiments, a step counter reset button 868 is provided to reset the step counter. In some embodiments, the step counter is reset by pressing a combination and/or sequence of buttons on the device. In some embodiments, the step counter is reset via a voice command.

In some embodiments, the cueing device 800 further comprises a voice instruction activation button 863. The voice instruction activation button may activate and deactivate the use of voice instructions. Voice instructions may comprise a vocal instructions synchronized with the set metronome rate to help a user maintain regular movement. Voice instructions may comprise instructions to overcome a movement irregularity or gait freezing episode. Voice instructions may be preloaded or custom recorded by a personal trainer or physician and uploaded to the device. In some embodiments, the cueing device comprises a voice command selections slider 861, allowing a user to choose between loaded voice commands or voice command sequences.

In some embodiment, the cueing device 800 comprises a belt clip 871. The belt clip may be removably coupled to the cueing device. In some embodiments, the belt clip comprises a first portion 873 and a second portion 875. The second portion may be biased toward the first portion to retain an article of clothing, belt, or the like between the first and second portions of the belt clip, thus retaining the cueing device to the article to which it is attached. The second portion may be biased toward the first portion by a spring, elastic member, or other suitable method.

In some embodiments, the cueing device further comprises a connection port 812. The connection port may be a universal serial bus (USB) or other suitable port for transferring data and/or power to and from the cueing device. In some embodiments, the connection port 812 allows a power source to be coupled to the internal battery. In an embodiment, battery indicator light 848 is provided to display the status of the battery and or device. The battery indicator light may have different modes to display activation of components of the cueing device and/or status of the charge for the internal battery. In some embodiments, the device comprises a power switch or button 832 which controls power to the entire device. The power switch may be activated to turn on the device. The power switch may be deactivated to turn off all components of the devices.

In some embodiments, the body of the cueing module is adjustable relative to the belt clip. In some embodiments, the portion of the belt clip which attaches to the body of the cueing module may be an adjustable hinge formed by a partially cylindrical surface with multiple indentations which are engaged by a locking tab. In some embodiments, the locking tab is provided on the belt clip. In some embodiments the locking tab is provided on the body of the cueing device. The adjustable angle may allow a user to adjust the distance of the projections emitted by laser 841 from their body.

In some embodiments, adjustment of the cueing module relative to the belt clip, or vice versa, will produce an audible click and/or tactile engagement. In some embodiments, wherein an adjustable hinge formed by a partially cylindrical surface with multiple indentations which are engaged by a locking tab is provided, the audible click and tactile engagement may be produced by the locking tab riding over the indentations and then engaging into the indentations due to the elasticity of the locking tab biasing the tab into the indentations. In some embodiments, wherein an engagement of two sets of teeth about a rotation of the belt hook may be enabled by two sets of teeth provided about the circumference of a radial surface.

In some embodiments, the adjustable angle between the belt hook and body of the cueing device is about 40 degrees to about 90 degrees. In some embodiments, the adjustable angle between the belt hook and body of the cueing device is about 40 degrees to about 50 degrees, about 40 degrees to about 60 degrees, about 40 degrees to about 70 degrees, about 40 degrees to about 80 degrees, about 40 degrees to about 90 degrees, about 50 degrees to about 60 degrees, about 50 degrees to about 70 degrees, about 50 degrees to about 80 degrees, about 50 degrees to about 90 degrees, about 60 degrees to about 70 degrees, about 60 degrees to about 80 degrees, about 60 degrees to about 90 degrees, about 70 degrees to about 80 degrees, about 70 degrees to about 90 degrees, or about 80 degrees to about 90 degrees. In some embodiments, the adjustable angle between the belt hook and body of the cueing device is about 40 degrees, about 50 degrees, about 60 degrees, about 70 degrees, about 80 degrees, or about 90 degrees. In some embodiments, the adjustable angle between the belt hook and body of the cueing device is at least about 40 degrees, about 50 degrees, about 60 degrees, about 70 degrees, or about 80 degrees. In some embodiments, the adjustable angle between the belt hook and body of the cueing device is at most about 50 degrees, about 60 degrees, about 70 degrees, about 80 degrees, or about 90 degrees.

In some embodiments, the metronome produces an audio signal which is output to the user at the rate which the metronome or device is set. The beat per minute rate of the metronome, as disclosed herein, may generally refer to the adjustable beat per minute rate of the whole device, as components of the device are synchronized in some embodiments. The rate of the metronome may be adjustable. The rate of the metronome may be a cadence rate. In some embodiments, the metronome rate is displayed in beats per minute (BPM). The metronome may produce audio cues at defined intervals of the metronome rate. For example, the audio cues may be produced at increments of quadruple (4), triple (3), double (2), whole (1), half (½), third (⅓), quarter (¼), eighths (⅛), sixteenths ( 1/16), thirty-second ( 1/32), sixty-eighth ( 1/64) or other suitable increments of the set beats per minute rate. In some embodiments the rate of the metronome is provided with a swing.

In some embodiments, the audible cue may comprise an accented beat. An accented beat may correspond to a directive for a user to take a step. For example, every other beat may be accented to direct the user to step on the accented beat.

In some embodiments, the range of the metronome rate is about 20 BPM to about 120 BPM. In some embodiments, the range of the metronome rate is about 20 BPM to about 40 BPM, about 20 BPM to about 60 BPM, about 20 BPM to about 80 BPM, about 20 BPM to about 100 BPM, about 20 BPM to about 120 BPM, about 40 BPM to about 60 BPM, about 40 BPM to about 80 BPM, about 40 BPM to about 100 BPM, about 40 BPM to about 120 BPM, about 60 BPM to about 80 BPM, about 60 BPM to about 100 BPM, about 60 BPM to about 120 BPM, about 80 BPM to about 100 BPM, about 80 BPM to about 120 BPM, or about 100 BPM to about 120 BPM. In some embodiments, the range of the metronome rate is about 20 BPM, about 40 BPM, about 60 BPM, about 80 BPM, about 100 BPM, or about 120 BPM. In some embodiments, the range of the metronome rate is at least about 20 BPM, about 40 BPM, about 60 BPM, about 80 BPM, or about 100 BPM. In some embodiments, the range of the metronome rate is at most about 40 BPM, about 60 BPM, about 80 BPM, about 100 BPM, or about 120 BPM.

In some embodiments, the metronome rate may be set by a metronome rate button. The metronome rate button may be depressed and the metronome rate will be set according to an interval between depressions. In some embodiments, the metronome rate button must be pressed at least twice to set the metronome rate.

In some embodiments, projection of the visual cues is synchronized with the metronome rate or the device rate. In some embodiments, projection of the visual cues may be projected at increments of the set beats per minute rate of the device or metronome. For example, the visual cues may be produced at increments of quadruple (4), triple (3), double (2), whole (1), half (½), third (⅓), quarter (¼), eighths (⅛), sixteenths ( 1/16), thirty-second ( 1/32), sixty-eighth ( 1/64) or other suitable increments of set beats per minute rate. In some embodiments, intervals of the projection of visual cues are synchronized with the metronome. The start and stop of a projection of a visual cue may be synchronized with the set beats per minute rate of the device.

In some embodiments, the cueing devices herein may further comprise a step counter or pedometer. The pedometer may be an accelerometer, wherein a step is counted when the measured acceleration surpasses a predetermined threshold. The accelerometer may be a micro-electromechanical (MEMS) accelerometer.

In some embodiments, the pedometer acts to calibrate the rate of the metronome. For example, a provided processing unit may count steps over a predetermined amount of time to set a corresponding BPM for the device. In some embodiments, a determination of distance per a predetermined amount of time sets the corresponding BPM for the device. In some embodiments, the predetermined time for calibrating the BPM is about 1 second to about 30 seconds. In some embodiments, the predetermined time for calibrating the BPM is about 1 second to about 5 seconds, about 1 second to about 10 seconds, about 1 second to about 15 seconds, about 1 second to about 20 seconds, about 1 second to about 25 seconds, about 1 second to about 30 seconds, about 5 seconds to about 10 seconds, about 5 seconds to about 15 seconds, about 5 seconds to about 20 seconds, about 5 seconds to about 25 seconds, about 5 seconds to about 30 seconds, about 10 seconds to about 15 seconds, about 10 seconds to about 20 seconds, about 10 seconds to about 25 seconds, about 10 seconds to about 30 seconds, about 15 seconds to about 20 seconds, about 15 seconds to about 25 seconds, about 15 seconds to about 30 seconds, about 20 seconds to about 25 seconds, about 20 seconds to about 30 seconds, or about 25 seconds to about 30 seconds. In some embodiments, the predetermined time for calibrating the BPM is about 1 second, about 5 seconds, about 10 seconds, about 15 seconds, about 20 seconds, about 25 seconds, or about 30 seconds. In some embodiments, the predetermined time for calibrating the BPM is at least about 1 second, about 5 seconds, about 10 seconds, about 15 seconds, about 20 seconds, or about 25 seconds. In some embodiments, the predetermined time for calibrating the BPM is at most about 5 seconds, about 10 seconds, about 15 seconds, about 20 seconds, about 25 seconds, or about 30 seconds. A manual reset switch or feature may be provided to initiate calibration or recalibrate the BPM. In some embodiments, recordation or sensing of one or more steps by the step counter or pedometer will automatically activate the visual and/or audio cues.

In some embodiments, a tilt switch is provided. The tilt switch may be provided to prevent accidental exposure of the light source into the eyes of a user or the eyes of persons within the vicinity of the user. The tilt switch may be configured to deactivate the light source when the angle of the projected light exceeds 65 degrees relative to a direction toward the ground (i.e. 65 degrees relative to experienced gravitational force). In some embodiments, the tilt switch may be configured to deactivate the light source when the angle of the projected light exceeds 60 degrees relative to a direction toward the ground. In some embodiments, the tilt switch may be configured to deactivate the light source when the angle of the projected light exceeds 45 degrees relative to a direction toward the ground. An alert signal may be provided in correlation with the deactivation of the light source due to an event which triggers the tilt switch. In some embodiments the tilt switch may be a MEMS gyroscope.

In some embodiments, the tilt switch is configured to deactivate the light source when the angle of the projected light relative to a direction toward the ground is about 45 degrees to about 90 degrees. In some embodiments, the tilt switch is configured to deactivate the light source when the angle of the projected light relative to a direction toward the ground is about 45 degrees to about 50 degrees, about 45 degrees to about 55 degrees, about 45 degrees to about 60 degrees, about 45 degrees to about 65 degrees, about 45 degrees to about 70 degrees, about 45 degrees to about 75 degrees, about 45 degrees to about 80 degrees, about 45 degrees to about 85 degrees, about 45 degrees to about 90 degrees, about 50 degrees to about 55 degrees, about 50 degrees to about 60 degrees, about 50 degrees to about 65 degrees, about 50 degrees to about 70 degrees, about 50 degrees to about 75 degrees, about 50 degrees to about 80 degrees, about 50 degrees to about 85 degrees, about 50 degrees to about 90 degrees, about 55 degrees to about 60 degrees, about 55 degrees to about 65 degrees, about 55 degrees to about 70 degrees, about 55 degrees to about 75 degrees, about 55 degrees to about 80 degrees, about 55 degrees to about 85 degrees, about 55 degrees to about 90 degrees, about 60 degrees to about 65 degrees, about 60 degrees to about 70 degrees, about 60 degrees to about 75 degrees, about 60 degrees to about 80 degrees, about 60 degrees to about 85 degrees, about 60 degrees to about 90 degrees, about 65 degrees to about 70 degrees, about 65 degrees to about 75 degrees, about 65 degrees to about 80 degrees, about 65 degrees to about 85 degrees, about 65 degrees to about 90 degrees, about 70 degrees to about 75 degrees, about 70 degrees to about 80 degrees, about 70 degrees to about 85 degrees, about 70 degrees to about 90 degrees, about 75 degrees to about 80 degrees, about 75 degrees to about 85 degrees, about 75 degrees to about 90 degrees, about 80 degrees to about 85 degrees, about 80 degrees to about 90 degrees, or about 85 degrees to about 90 degrees. In some embodiments, the tilt switch is configured to deactivate the light source when the angle of the projected light relative to a direction toward the ground is about 45 degrees, about 50 degrees, about 55 degrees, about 60 degrees, about 65 degrees, about 70 degrees, about 75 degrees, about 80 degrees, about 85 degrees, or about 90 degrees. In some embodiments, the tilt switch is configured to deactivate the light source when the angle of the projected light relative to a direction toward the ground is at least about 45 degrees, about 50 degrees, about 55 degrees, about 60 degrees, about 65 degrees, about 70 degrees, about 75 degrees, about 80 degrees, or about 85 degrees. In some embodiments, the tilt switch is configured to deactivate the light source when the angle of the projected light relative to a direction toward the ground is at most about 50 degrees, about 55 degrees, about 60 degrees, about 65 degrees, about 70 degrees, about 75 degrees, about 80 degrees, about 85 degrees, or about 90 degrees.

In some embodiments, a fall alert may be configured to transmit an alert when a fall is detected. The detection of a fall may be activated by a sudden change in angle or acceleration associated with the user experiencing a fall. The sudden change in acceleration or angle at which the device is tilted may be measured by an accelerometer or gyroscope. The associated accelerometer or gyroscope may be provided as part of the cueing device or other systems disclosed herein. The associated accelerometer or gyroscope on an external device such as a personal mobile device or wearable device.

In some embodiments, the audio cues produced may be provided in the form of a note or tone. In some embodiments, the audio cues may be provided as a voice instruction. In some embodiments, upon detection of a freezing of gait or irregular movement event a set of voice instructions may be initiated. The set of voice instructions may include instructions to guide the user in resuming a normal movement.

In some embodiments, if the movement correction system does not detect a step or detected movement after a provided cue, the device will register an irregular movement or freezing event. In some embodiments, registration of an irregular movement or freezing event initiates an instruction sequence. The instruction sequence may provide a sequence of cues to help the user resume regular movement. The sequence of cues of the instruction sequence may be a sequence of voice instructions. In some embodiments, registration of an irregular movement or freezing event initiates projection of visual cues. In some embodiments, registration of an irregular movement or freezing event initiates audio cues.

In some embodiments, irregular movement or freezing detection may be a programmable feature. The option to detect an irregular movement event may be activated or deactivated through user engagement provided by a switch, dial, and/or interactive user interface. The option to receive an instruction sequence following an irregular movement event may be activated or deactivated through user engagement provided by a switch, dial, and/or interactive user interface.

In some embodiments, the system further comprises a microphone. The microphone may be provided to receive commands from the user to change settings of the cueing device and movement correction system. In some embodiments, the metronome rate may be set using a voice command. The microphone may determine and interval between at least two audio event peaks to calibrate the metronome rate. In some embodiments, after receiving a voice command to set the metronome rate, a period of time passes before the microphone may sense the at least two audio events which are used to calibrate the metronome rate. The period of time which passes between the command and the audio events may be about a half-second to about 2 seconds.

In some embodiments, the system further comprises a digital processing device comprising one or more central processing units (CPUs), a memory, and/or a communication module. The memory may include random-access memory, read-only memory, and or flash memory. The communication module may include a wireless interface for wirelessly transmitting data collected by the system to an external device. The communication module may include a hardwire interface for transmitting data collected from the system to an external device via a wired connection. The external device may comprise a digital processing device configured to aggregate data collected by and received from the movement correction system.

Data collected by the system may include number of steps taken, time of steps taken, duration of walking events, number of irregular movement events recorded, time elapsed, distance travelled, falls recorded, and other datum which may be correlated with movement of a user.

In some embodiments, irregular movement events may include step asymmetry. In some embodiments, steps measured by the accelerometer or pedometer are compared to the compared the metronome cadence. Steps taken at irregular intervals may be used to analyze the step symmetry of the patient. The detected intervals of steps taken may be compared to the metronome cadence and stored or recorded by the system. Data which includes differences between the steps taken and the metronome cadence may be analyzed over time to assess improvement or regression of a patient or user utilizing the system. In some embodiments, detected intervals of steps taken are compared accent notes of the cadence which represent the audio cue at which a patient or user should take the step.

Optionally, in some embodiments, the platforms, systems, media, and methods described herein include a digital processing device, or use of the same. In further embodiments, the digital processing device includes one or more hardware central processing units (CPUs) or general purpose graphics processing units (GPUs) that carry oust the device's functions. In still further embodiments, the digital processing device further comprises an operating system configured to perform executable instructions. Optionally, in some embodiments, the digital processing device is optionally connected a computer network. In further embodiments, the digital processing device is optionally connected to the Internet such that it accesses the World Wide Web. In still further embodiments, the digital processing device is optionally connected to a cloud computing infrastructure. In other embodiments, the digital processing device is optionally connected to an intranet. In other embodiments, the digital processing device is optionally connected to a data storage device.

In accordance with the description herein, suitable digital processing devices include, by way of non-limiting examples, server computers, desktop computers, laptop computers, notebook computers, sub-notebook computers, netbook computers, netpad computers, set-top computers, and media streaming devices, handheld computers, Internet appliances, mobile smartphones, tablet computers, personal digital assistants, video game consoles, and vehicles. Those of skill in the art will recognize that many smartphones are suitable for use in the system described herein. Those of skill in the art will also recognize that select televisions, video players, and digital music players with optional computer network connectivity are suitable for use in the system described herein. Suitable tablet computers include those with booklet, slate, and convertible configurations, known to those of skill in the art.

Optionally, in some embodiments, the digital processing device includes an operating system configured to perform executable instructions. The operating system is, for example, software, including programs and data, which manages the device's hardware and provides services for execution of applications. Those of skill in the art will recognize that suitable server operating systems include, by way of non-limiting examples, FreeBSD, OpenBSD, NetBSD®, Linux, Apple® Mac OS X Server®, Oracle® Solaris®, Windows Server®, and Novell® NetWare®. Those of skill in the art will recognize that suitable personal computer operating systems include, by way of non-limiting examples, Microsoft® Windows®, Apple® Mac OS X®, UNIX®, and UNIX-like operating systems such as GNU/Linux®. Optionally, in some embodiments, the operating system is provided by cloud computing. Those of skill in the art will also recognize that suitable mobile smart phone operating systems include, by way of non-limiting examples, Nokia® Symbian® OS, Apple® iOS®, Research In Motion® BlackBerry OS®, Google® Android®, Microsoft® Windows Phone® OS, Microsoft® Windows Mobile® OS, Linux®, and Palm® WebOS®. Those of skill in the art will also recognize that suitable media streaming device operating systems include, by way of non-limiting examples, Apple TV®, Roku®, Boxee®, Google TV®, Google Chromecast®, Amazon Fire®, and Samsung® HomeSync®. Those of skill in the art will also recognize that suitable video game console operating systems include, by way of non-limiting examples, Sony® PS3®, Sony® PS4®, Microsoft® Xbox 360®, Microsoft Xbox One, Nintendo® Wii®, Nintendo® Wii U®, and Ouya®.

Optionally, in some embodiments, the device includes a storage and/or memory device. The storage and/or memory device is one or more physical apparatuses used to store data or programs on a temporary or permanent basis. Optionally, in some embodiments, the device is volatile memory and requires power to maintain stored information. Optionally, in some embodiments, the device is non-volatile memory and retains stored information when the digital processing device is not powered. In further embodiments, the non-volatile memory comprises flash memory. Optionally, in some embodiments, the non-volatile memory comprises dynamic random-access memory (DRAM). Optionally, in some embodiments, the non-volatile memory comprises ferroelectric random access memory (FRAM). Optionally, in some embodiments, the non-volatile memory comprises phase-change random access memory (PRAM). In other embodiments, the device is a storage device including, by way of non-limiting examples, CD-ROMs, DVDs, flash memory devices, magnetic disk drives, magnetic tapes drives, optical disk drives, and cloud computing based storage. In further embodiments, the storage and/or memory device is a combination of devices such as those disclosed herein.

Optionally, in some embodiments, the digital processing device includes a display to send visual information to a user. Optionally, in some embodiments, the display is a liquid crystal display (LCD). In further embodiments, the display is a thin film transistor liquid crystal display (TFT-LCD). Optionally, in some embodiments, the display is an organic light emitting diode (OLED) display. In various further embodiments, on OLED display is a passive-matrix OLED (PMOLED) or active-matrix OLED (AMOLED) display. Optionally, in some embodiments, the display is a plasma display. In other embodiments, the display is a video projector. In yet other embodiments, the display is a head-mounted display in communication with the digital processing device, such as a VR headset. In further embodiments, suitable VR headsets include, by way of non-limiting examples, HTC Vive, Oculus Rift, Samsung Gear VR, Microsoft HoloLens, Razer OSVR, FOVE VR, Zeiss VR One, Avegant Glyph, Freefly VR headset, and the like. In still further embodiments, the display is a combination of devices such as those disclosed herein.

Optionally, in some embodiments, the digital processing device includes an input device to receive information from a user. Optionally, in some embodiments, the input device is a keyboard. Optionally, in some embodiments, the input device is a pointing device including, by way of non-limiting examples, a mouse, trackball, track pad, joystick, game controller, or stylus. Optionally, in some embodiments, the input device is a touch screen or a multi-touch screen. In other embodiments, the input device is a microphone to capture voice or other sound input. In other embodiments, the input device is a video camera or other sensor to capture motion or visual input. In further embodiments, the input device is a Kinect, Leap Motion, or the like. In still further embodiments, the input device is a combination of devices such as those disclosed herein.

Any methods disclosed herein comprise one or more steps or actions for performing the described method. The method steps and/or actions may be interchanged with one another. In other words, unless a specific order of steps or actions is required for proper operation of the embodiment, the order and/or use of specific steps and/or actions may be modified.

### I. Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Embodiments may be understood by reference to the drawings, wherein like parts are designated by like numerals throughout. It will be readily understood by one of ordinary skill in the art having the benefit of this disclosure that the components of the embodiments, as generally described and illustrated in the figures herein, could be arranged and designed in a wide variety of different configurations. Thus, the following more detailed description of various embodiments, as represented in the figures, is not intended to limit the scope of the disclosure, but is merely representative of various embodiments. While the various aspects of the embodiments are presented in drawings, the drawings are not necessarily drawn to scale unless specifically indicated.

It will be appreciated that various features are sometimes grouped together in a single embodiment, figure, or description thereof for the purpose of streamlining the disclosure. Many of these features may be used alone and/or in combination with one another.

The phrases “coupled to” and “in communication with” refer to any form of interaction between two or more entities, including mechanical, electrical, magnetic, electromagnetic, fluid, and thermal interaction. Two components may be coupled to or in communication with each other even though they are not in direct contact with each other. For example, two components may be coupled to or in communication with each other through an intermediate component.

The directional terms “distal” and “proximal” are given their ordinary meaning in the art. That is, the distal end of a medical device means the end of the device furthest from the practitioner or user during use. The proximal end refers to the opposite end, or the end nearest the practitioner during use. As specifically applied to the cueing module of a cueing device, the proximal end of the cueing module refers to the end away from the walking surface and the distal end refers to the opposite end, the end nearest the walking surface.

The term body should be understood as the outline formed by the outer surface(s) of a component, part, device, apparatus, module, or the like.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

The terms “subject,” “individual,” or “patient” are often used interchangeably herein. A “subject” can be a biological entity containing expressed genetic materials. The biological entity can be a plant, animal, or microorganism, including, for example, bacteria, viruses, fungi, and protozoa. The subject can be tissues, cells and their progeny of a biological entity obtained in vivo or cultured in vitro. The subject can be a mammal. The mammal can be a human. The subject may be diagnosed or suspected of being at high risk for a disease. In some cases, the subject is not necessarily diagnosed or suspected of being at high risk for the disease.

As used herein, the term “about” a number refers to that number plus or minus 10% of that number. The term “about” a range refers to that range minus 10% of its lowest value and plus 10% of its greatest value.

The section headings used herein are for organizational purposes only and are not to be construed as limiting the subject matter described.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A device for treatment of a movement disorder, the device comprising:
a metronome having a rate; and
a light source configured to project on an external surface.

2. The device of claim 1, further comprising an accelerometer.

3. The device of claim 2, wherein the accelerometer is configured as a step counter.

4. The device of claim 3, wherein a ratio of a number of steps recorded by the step counter over a predetermined interval of time is used to calibrate the rate of the metronome.

5. The device of claim 2, wherein a fall event is registered when the accelerometer senses an acceleration which exceeds a predetermined threshold.

6. The device of claim 5, wherein an alert is produced subsequent to the fall event being registered.

7. The device of claim 6, wherein the device further comprises at least one audio transducer, and the alert comprises an auditory signal emitted from the at least one audio transducer.

8. The device of claim 1, further comprising a belt clip.

9. The device of claim 8, wherein the belt clip comprises a first portion and a second portion, wherein the second portion is biased towards the first portion to retain the device onto an article worn by a user.

10. The device of claim 9, wherein the second portion is biased towards the first portion by a spring.

11. The device of claim 8, wherein the belt clip is rotatably relative to a body of the device.

12. The device of claim 11, wherein a first mating disposed on the body of the device comprises a plurality of teeth to engages a second mating surface disposed on the clip to provide a rotatable engagement allowing for selective positioning of the body of the device relative to the clip.

13. The device of claim 12, wherein the second mating surface comprises a plurality of teeth to engage with the plurality of teeth of the first mating surface.

14. The device of claim 12 or 13, wherein the plurality of teeth of the first mating surface are provided on a circular protrusion and about an axis of rotation by which the clip rotates relative to the body of the device.

15. The device of claim 8 or 11, wherein an angle between the belt clip and a body of the device is adjustable.

16. The device of claim 15, wherein a means for adjusting the angle between the belt clip and a body of the device comprises an adjustable hinge formed by a partially cylindrical surface having a plurality indentations which are engaged by a locking tab.

17. The device of claim 1, further comprising a communication module configured to transmit audio signals from the metronome to an external audio device.

18. The device of claim 17, wherein the external audio device comprises wireless hearing aids.

19. The device of claim 17, wherein the external audio device comprises wireless headphones.

20. The device of claim 1, further comprising a microphone, wherein the microphone is configured to receive voice commands from a user to changes settings of the device.

21. The device of claim 20, wherein the rate of the metronome is determined by an elapsed time between peaks of at least two audio events received by the microphone.

22. The device of claim 1, wherein the external surface is a walking surface.

23. The device of claim 1, wherein the light source is a laser.

24. The device of claim 23, wherein the laser is provided on a rotatable platform.

25. The device of claim 23 or 24, wherein the laser is configured to project one or more lines onto the external surface.

26. The device of claim 25, wherein in the one or more lines are configured to be perpendicular to the line of site of a body of the cueing device.

27. The device of claim 26, wherein the external surface is a walking surface.

28. The device of claim 1 or 23, further comprising a tilt switch configured to deactivate the light source when the device is angled such that a projection of the light source exceeds a predetermined angle relative to a direction of experienced gravitational force.

29. The device of claim 28, wherein the tilt switch comprises a gyroscopic sensor.

30. The device of claim 28, wherein the predetermined angle is 60 degrees.

31. The device of claim 1, further comprising a volume control.

32. The device of claim 31, wherein the volume control is a potentiometer.

33. The device of claim 31, wherein the volume control comprises two or more buttons.

34. The device of claim 1, further comprising a metronome rate control.

35. The device of claim 34, wherein the metronome rate control is a potentiometer.

36. The device of claim 34, wherein the metronome rate control comprises two or more buttons.

37. A method of preventing an irregular movement in a user, the method comprising:
setting a rate of a metronome;
emitting an audio signal to the user, the audio signal corresponding with the rate of the metronome; and
projecting a visual cue onto a walking surface in front of the user.

38. The method of claim 37, further comprising a step of counting steps of the user over a predetermined period of time, and wherein the step of setting the rate of the metronome further comprises automatically setting the rate of the metronome based on a number of steps counted over the predetermined period of time.

39. The method of claim 38, further comprising recording a time elapsed since recordation of a step and recording an irregular movement event if the time lapsed exceeds a predetermined elapsed.

40. The method of claim 39, further comprising a step of resetting the rate of the metronome subsequent to recording the irregular movement event.

41. The method of claim 39, further comprising a step of emitting a sequence of audio signals subsequent to recording the irregular movement event.

42. The method of claim 40, wherein the sequence of audio signals comprises one or more voice instructions directed to assisting the user in overcoming the irregular movement event.

43. The method of claim 37, wherein the step of emitting an audio signal to the user further comprises wirelessly transmitting the audio signal to an audio device.

44. The method of claim 37, further comprising receiving one or more voice commands.

45. The method of claim 44, wherein the step of setting the rate of the metronome is based upon receiving the one or more voice commands.

46. The method of claim 45, wherein the step of setting the rate of the metronome further comprises analyzing a time elapsed between peaks of at least two audio signals received and setting the rate of the metronome to correspond to elapsed time between the at least two audio signals received.
